(19) United States
(12) Patent Application Publication
(10) Pub. No.: US 2022/0147944 A1
(43) Pub. Date: May 12, 2022

(54) A METHOD OF IDENTIFYING AND ADDRESSING CLIENT PROBLEMS

(71) Applicants: Cognitive Industries Pty Ltd, Sydney, NSW (AU); Valise.TV Pty Ltd, Sydney, NSW (AU)

(72) Inventors: Simon Kendall, Bradbury, New South Wales (AU); Jamie Carroll, Camperdown, New South Wales (AU)

(21) Appl. No.: 17/438,554

(22) PCT Filed: Mar. 17, 2020
(86) PCT No.: PCT/AU2020/050254

§ 371 Date: Sep. 13, 2021

(30) Foreign Application Priority Data
Mar. 18, 2019 (AU) 2019900888

(51) Int. Cl. G06Q 10/10; G06V 20/40; G06V 40/20; G06F 40/35; H04L 51/02

(52) U.S. Cl. CPC G06Q 10/105; G06V 20/41; G10L 25/63; G06F 40/35; H04L 51/02; G06V 40/20

(57) ABSTRACT

A method of identifying and addressing client problems, comprising the steps of: —using a chat bot to ask a human first client a series of questions and to receive the clients answers; —computer processing the answers to identify: —that the client has experienced a problem; and—what solution the client implemented to solve that problem; —using the chat bot to ask a human second client a series of questions and to receive that client's answers; —computer processing the second clients answers to determine that that client has substantially the same problem that the first client had; and delivering substantially the first clients solution to the second client.

### FIELD OF INVENTION

A preferred form of this invention relates to a method of identifying and addressing staff problems within an organisation.

### BACKGROUND

Inefficiencies can be experienced by business and other organisations, particularly where they have many clients (eg staff or volunteers) and such people, on different occasions, experience the same problem in the course of doing their work. Often one client has solved a problem but the knowledge of how that was achieved is not shared. This can lead to wastage of time in that the next client with the same problem does not know about the solution and has to solve the problem from scratch.

### OBJECT OF THE INVENTION

It is an object of a preferred embodiment of the invention to go at least some way towards addressing the above issue. While this applies to preferred embodiments, it should be understood that the object of the invention per se is simply to provide the public with a useful choice. Therefore, any objects or benefits applicable to preferred embodiments should not be taken as a limitation on the scope of any claims expressed more broadly.

### Definitions

The terms “comprises” or “comprising” or derivatives thereof should not be interpreted as limiting. For example, if used in relation to a combination of features they should be taken to mean that optionally, but not necessarily, there may be additional features that have not been mentioned.

### SUMMARY OF THE INVENTION

According to one aspect of the invention there is provided a method of identifying and addressing client (eg staff) problems, comprising the steps of:
using a chat bot to ask a human first client a series of questions and to receive the client's answers;
computer processing the answers to identify:
   that the client has experienced a problem; and
   what solution the client implemented to solve that problem;
using the chat bot to ask a human second client a series of questions and to receive that client's answers;
computer processing the second client's answers to determine that that client has substantially the same problem that the first client had; and
delivering substantially the first client's solution to the second client.

Optionally the chat bot asks the clients questions pertaining to their wellbeing and determines, based on their answers, when they have a similar problem relating to their wellbeing.

Optionally the problem, when related to wellbeing, is that the client is feeling at least one of:
over-worked;
underutilised;
under-valued;
unappreciated;
pressured;
anxious;
worried;
unknowledgeable;
in need of training;
victimised; and
bullied.

Optionally the chat bot determines the emotional or mental disposition of the clients based on the answers they give to the questions (eg in terms of statements made or audio tone, etc).

Optionally the chat bot receives video image data from communications devices used by the clients (eg computers, tablets, phones) and, based on such data, determines the emotional or mental disposition of the clients (eg based on bodily (eg facial) movements or gestures).

Optionally the client answers, or the video imagery data, are computer processed to determine whether the client in each case is one or more of:
surprised;
confused;
anxious;
agitated;
annoyed;
angry;
sad;
happy;
pleased; and
satisfied.

Optionally the chat bot communicates with the client in a manner sympathetic to the clients' emotional or mental disposition as determined above.

Optionally the answers are computer processed to determine the level or performance of the clients and/or of the organisation they are engaged in.

Optionally the computer system:
a) records personal circumstances experienced by the clients and communicated to the system; and
b) chats with the clients in sympathy with such circumstances.

Optionally the computer system:
a) identifies ‘likes’ and ‘dislikes’ communicated by the clients via social media platform accounts of those clients; and
b) chats with the clients in sympathy with the likes and dislikes they expressed.

### DRAWINGS

Some preferred embodiments of the invention will now be described by way of example and with reference to the accompanying drawing(s), of which:

FIG. 1 is a conceptual illustration of a method of identifying and addressing staff problems within an organisation;

FIG. 2 illustrates detail of the system, including an algorithm for processing client queries;

FIG. 3 illustrates further detail of the system, including a ‘to do’ task setting and reminder routine;

FIG. 4 illustrates still further detail of the system, including a routine for determining the ‘wellness’ of clients;

FIG. 5 illustrates a portion of the system programming for controlling wellness inquiries;

FIG. 6 illustrates a portion of the system that operates in sympathy to the personality of the client; and

FIG. 7 illustrates a preferred portion of the system that operates in sympathy with social media likes and dislikes posted by the clients.

### DETAILED DESCRIPTION

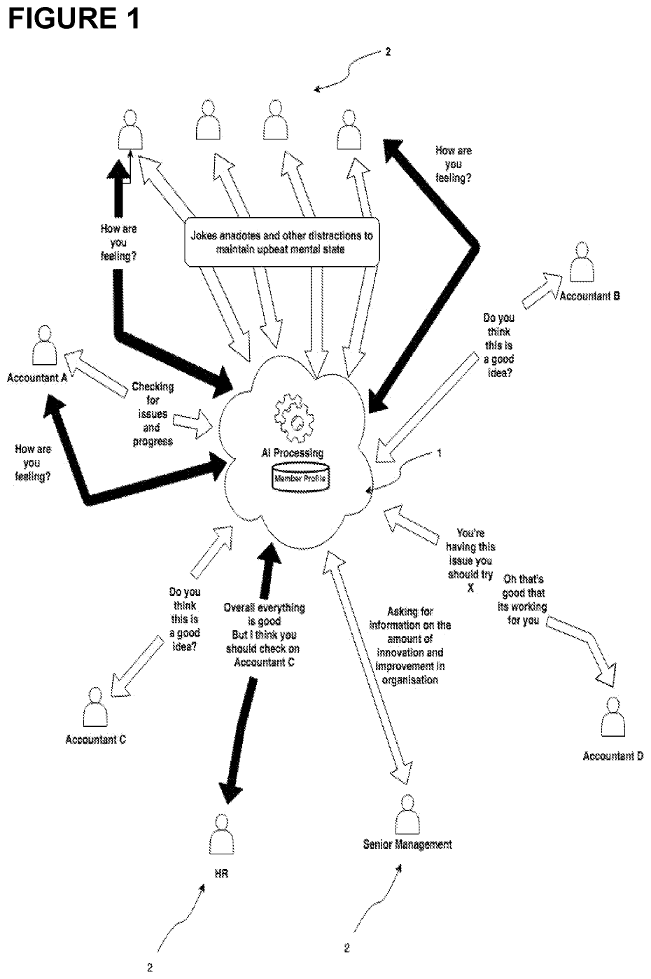

Referring to FIG. 1, a computer system 1 is used by a business organisation to manage its affairs. The system 1 incorporates a chat bot that interacts with client employees 2 of the organisation. In FIG. 1 each image of a human represents a different one of the employees. The term ‘chat bot’ is used generically in this document and comprises a software routine that is able to ‘chat dialogue’ with a client interactively. Preferably the chat bot operates in the manner of a ‘virtual assistant’, being programmed to learn and record information about human user clients based on current or past dialogue with them. The ‘learned’ information is used by the computer system 1 for future interactions with the same or different clients. Preferably the computer system 1 is programmed to learn in a linear/circular manner rather than a tree-branch manner.

One function of the chat bot is to ask at least certain of the employees questions to identify whether they have experienced any problems in the course of their work. The employees engage with the chat bot online, using their computers (eg via intranet or the internet), by typing text, voice messaging and/or video imaging. The chat bot dialogues with the employees using one or more of the same media. For example the chat bot may communicate with onscreen text, a voice playing to the employee or a video played with voice and imagery.

As the employee is dialoguing with the chat bot, the system 1 determines the emotional or mental disposition of the employee and tailors chat bot communications in sympathy with this. For example if an employee types messages, displays audio tone or bodily movements (as detected via their computer's camera and microphone) that indicate frustration or anger, then the chat bot uses more ‘understanding’ dialogue in response.

The chat bot may, solicited or unsolicited by each employee, ask the employee a series of questions designed to identify whether the employee has encountered a problem, and if so then what it was. The chat bot also asks the employee to communicate how the problem was solved. The system records both the problem and the solution.

In cases where the chat bot identifies that another employee subsequently has the same problem and has not solved it, then the chat bot communicates to this employee what the solution was. In this way the second employee is able to take advantage of the work done by the first employee in solving the problem. This saves time and resources as the second employee does not have to come up with a solution on his or her own.

In some embodiments of the invention the system, via the chat bot, identifies that an employee has a wellness issue. For example the employee may be feeling one or more of:
over-worked;
underutilised;
under-valued;
unappreciated;
pressured;
anxious;
worried;
unknowledgeable;
in need of training;
victimised; and
bullied,
etc.

The employee's lack of well-being, and what the nature of this is, is identified by the system as a problem. If the issue is determined by the system 1 to be minor then the chat bot may communicate a solution to the employee, selected from a list of pre-recorded solutions for the same problem. Such solutions may be system learned, eg through chat bot communications with other employees, or loaded into the system 1 by the business. If the system determines that the employee has a significant wellness problem then it is preferably indicated to a human administrator or supervisor so that the matter can be addressed on more of a human level.

If the system 1 determines that many employees within a business have a wellness problem, especially if it is the same problem, then this is recorded and communicated by the system to management or human resources personnel for human investigation. This assists the business to effectively manage employee relations and identify possible morale and other problems early on.

In some embodiments of the invention the system 1 shares work projects among a group of employees, and the chat bot questions them to obtain feedback on possible problems concerning the project and suggested solutions to these. The solutions may be shared by the system among all members of the group, via the chat bot, or in another way, eg by email, etc.

Referring again to FIG. 1, the messages between employees and the system 1 are examples of computer delivered text messages between the two. As can be seen, the topics of dialogue may be quite varied.

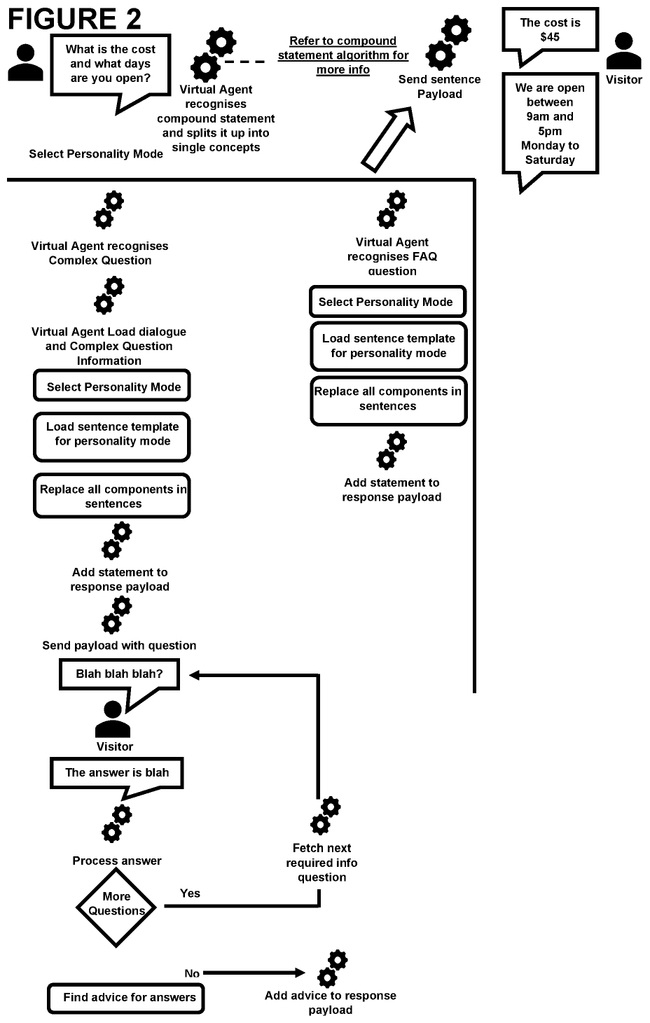

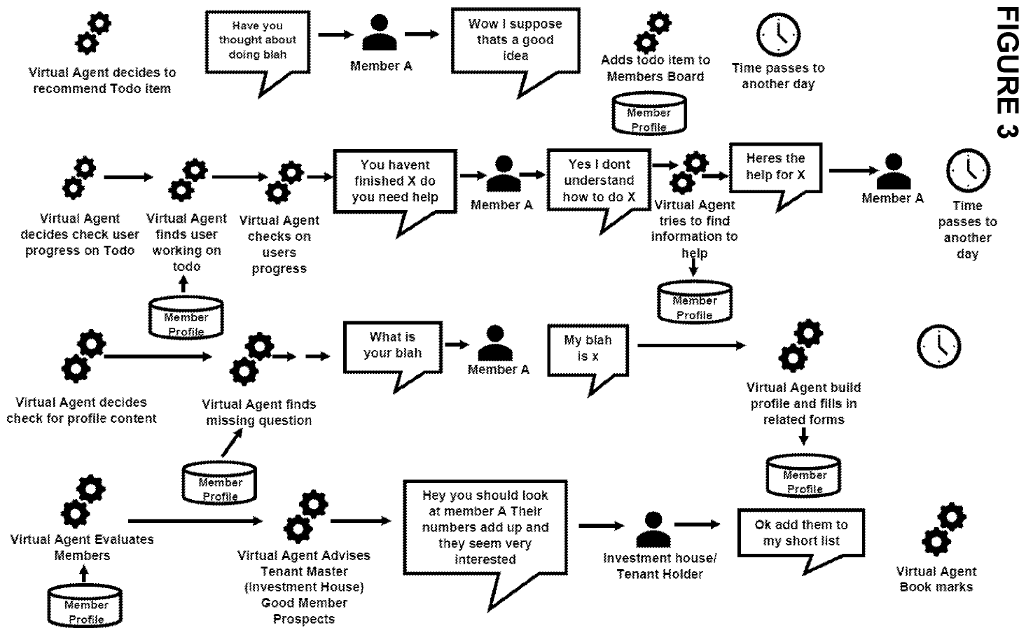

Referring to FIG. 2, in at least some preferred embodiments of the invention the system 1 computer processes sentences communicated by each employee to detect queries. A query is detected by identifying a query ‘opener’ term such as “how” or “where”, and query ‘ending’ terms such as “?” and “!”. The system 1 also interrogates the employee sentences for compound queries, for example as indicated by ‘joining’ words such as “and” and “but”. Based on the presence of ‘opening’, ‘closing’ and ‘joining’ terms the system divides the sentences into a collection of separate queries or sub-queries, and processes these. They may then be answered to the employee by the chat bot sequentially, or in any order deemed to be most appropriate by the system 1.

As also illustrated in FIG. 2, the system determines the personality type of employees dialoguing with the chat bot. If for example an employee speaks formally, and thereby indicates a more formal personality type or just a preference to dialogue formally, then the chat bot also dialogues in formal language. Conversely, the chat bot uses more casual language if the system determines that the employee is speaking casually and therefore has a more casual personality or just a preference to dialogue casually.

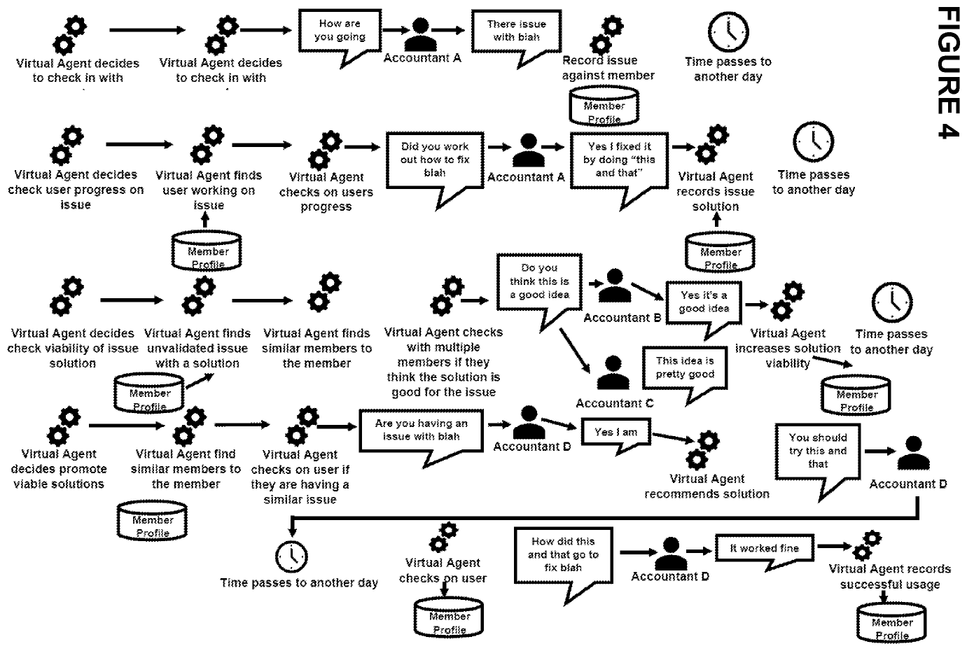

Referring to FIG. 3, in some embodiments of the invention the system 1 incorporates a ‘virtual agent’ software algorithm for interacting with the employees by way of the chat bot. For example, as shown in the first line of FIG. 3, the virtual agent has the chat bot recommend a ‘to do’ task to an employee. The employee agrees to or otherwise accepts the task and the virtual agent then adds a record of the task to a data file referenced to the employee concerned. As shown on line 2, after a period of time the virtual agent checks on the system to see whether the task has been actioned. If it has not then the virtual agent issues a reminder communication to the employee. The employee replies with a message communicating that the employee does not know how to do or complete the task. The virtual agent searches the system for any data records pertaining to solutions that other employees have used for the same or a similar task and communicates the solution to the employee.

Referring to the third line of FIG. 3, the same or another virtual agent searches system data records and determines that an employee profile has missing or otherwise sub-optimal information. The virtual agent communicates a query to the employee asking for the information. The information is received via the chat bot and added by the agent to the employee's profile.

Referring to the fourth line of FIG. 3, the same or another virtual agent evaluates the profiles of employees or other ‘members’ to the system. From this it is determined that a particular member fits a system profile for a person needing ‘such and such’ assistance, product or service. The system adds the person to a list of candidates for follow-up and communicates details for the candidate to an employee tasked with making contact. Alternatively the chat bot may contact the candidate directly.

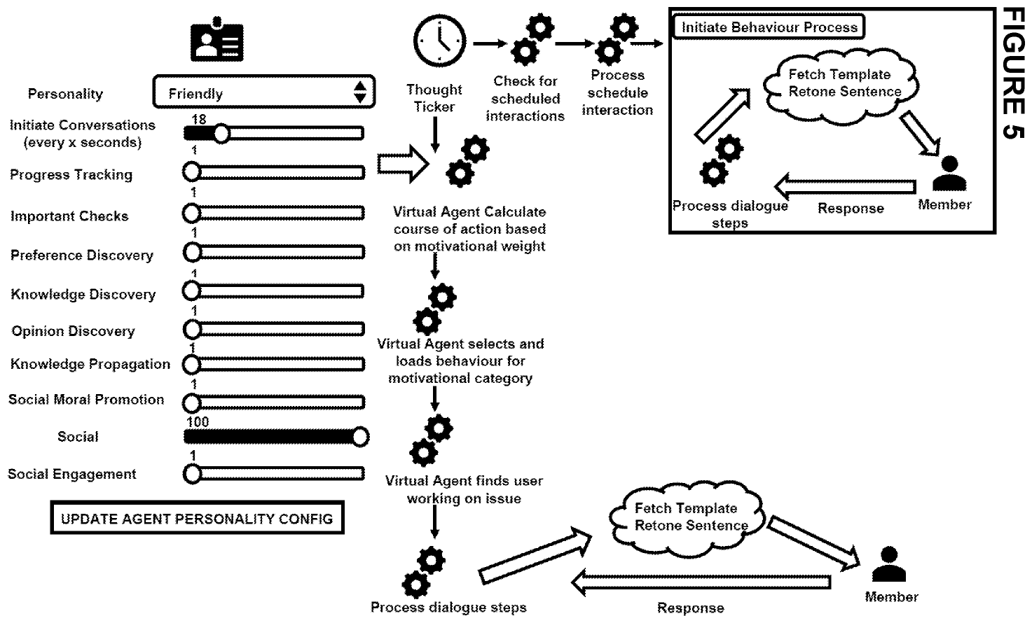

FIG. 4 illustrates a ‘wellness’ software algorithm according to a preferred embodiment of the invention. As shown in the first line of FIG. 4, a virtual agent software routine periodically checks in on the well-being of an employee or other member of the system via the chat bot. The member communicates an issue they are grappling with and the system makes a data record of this. Referring to the second line, the virtual agent later checks on the same member via the chat bot to see how they are going. The member reports that they devised or found a solution to the issue and communicates that solution via the chat bot. A data record of the solution is made in the system. Referring to the third line, the system then communicates the issue and the solution to other members of the system, again via the chat bot, to see whether they agree that the solution is a good one. Referring to the fourth and fifth lines, if the feedback on the solution is positive then the system promotes the solution to other members via the chat bot.

FIG. 5 illustrates a portion of the system programming for controlling wellness inquiries. As indicated at 3, the system has human adjustable motivation weighting settings. More specifically, the drawing illustrates a cognitive recurring process that emulates human ‘free will processing’. The step—“Virtual Agent Calculate course of action Based on Motivational Weighting” retrieves the configuration of weightings for a client employee relating to categories of behaviour from a system account for the person. Based on the weighting the system calculates what system communications to issue. Notionally this simulates what people do when they ‘choose what they are doing next’. For example, people will superficially pick a course of action based on what they like. If they like ‘Option A’ over ‘Option B’ then they will be more likely to pick ‘Option A’. With a behaviour category calculated, the system randomly selects a behaviour definition (eg type of communication) associated with that category that is more likely to appeal to the person.

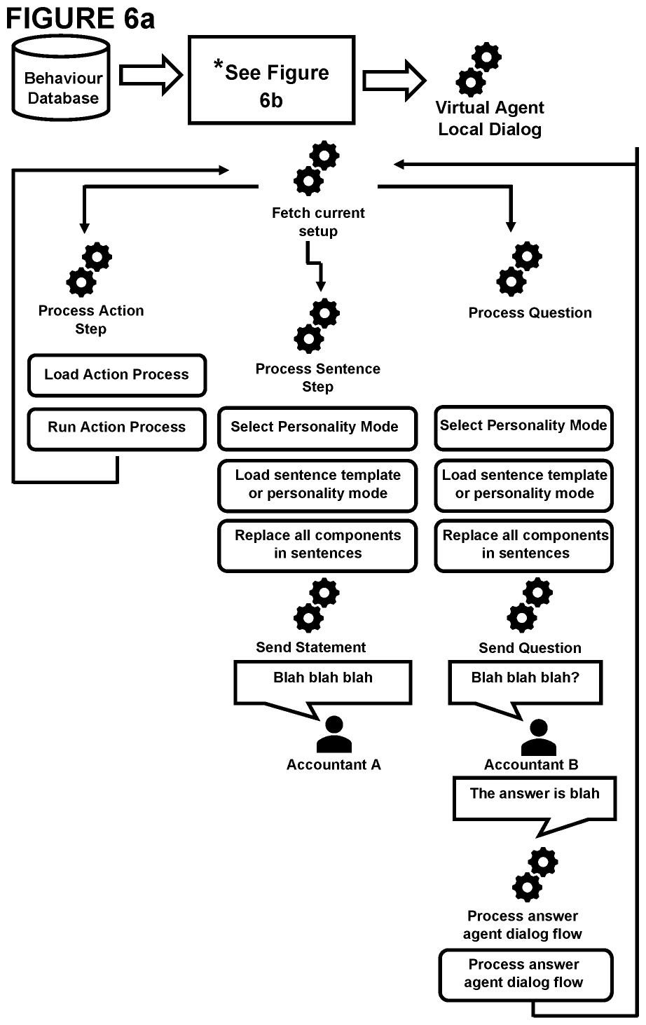

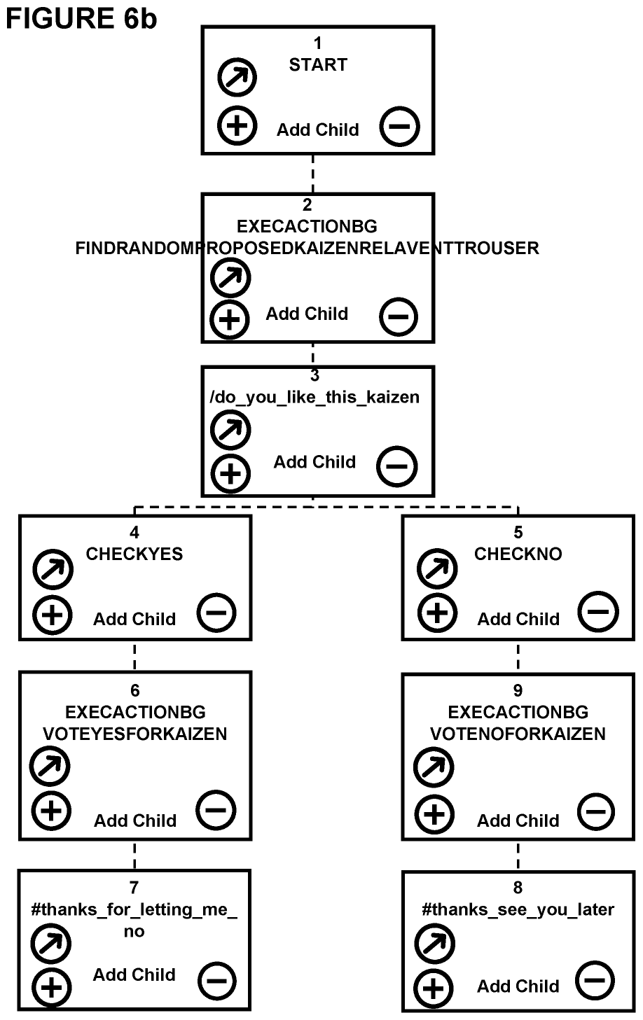

FIG. 6 illustrates a portion of the system that operates in sympathy to the personality of the client employee speaking with the chat bot. More specifically in the ‘Virtual Agent Load Dialog’ step the system loads the most recent behaviour definition for use with the current conversation. With the definition loaded the system then determines what the current step is in the behaviour concerned, and just what sort of process it is. This is a looped process where the system will only exit the loop when it needs information from the person the chat bot is conversing with.

When the step to be taken is an ‘action’ the system references an action definition database, loads an action code, and then executes that code. The code is soft coded not hard coded. Due to this there is a capacity for the system to create its own actions based on previous experience, research and conversations carried out by the system via the chat bot. By way of explanation, ‘soft coding’ refers to obtaining a value or function from some external resource, such as a pre-processor macro, external constant, configuration file, command line argument or database table. In the context of this document, soft coding refers to programming like instructions that are stored in a database and not in compiled executable code. Soft coding is the opposite of hardcoding, which refers to coding values and functions in source code. Hard Coding refers to computer code that is predefined by software developers that can only be modified compiled and then released

With further reference to FIG. 6, when the step is a sentence the system calculates a personality mode to use based on a default client setting and contextually on who the chat bot is talking to. With the personality mode determined the system then calculates what set of sentence templates to use (based on a sentence key and personality mode). The system randomly selects a sentence from the set and does the required replacement of context values. For example, a concept that is being discussed, the person's name or values retrieved in a previous action step, etc. With the response calculated the system appends it to the responses that will be returned when the process returns to the person the chat bot is talking to.

The last step is a ‘question’. The system drives the chat bot to go through the same process as if it was a sentence step to calculate what to say to the person concerned. But instead of merely appending this to the response it will rather append it to the response and then wait for a response to the question. When the system gets a response it will evaluate whether the response is valid and, if not, then it will re-ask the question while letting the person know what was wrong with the answer. When the system receives a valid response it will continue with the behaviour definition process.

### Virtual Assistant Social Presence

In preferred forms of the invention the chart bot, functioning as a virtual assistant, is programmed to use social interaction to build a notional relationship or rapport with human clients. It does so by expressing emotional states and opinions to human clients. For example, it communicates ‘like’ or ‘dislike’ statements in response to activities or statements by clients or other human users. For example, if the computer system 1 learns that a client has an interest in asteroids, the system will periodically research that topic and talk about it when interacting with the client; for example in a post via a custom social network platform or one of the more common platforms such as Twitter. This gives the virtual assistant to have a notional or client perceived richer or more human character. The virtual assistant in a sense interweaves ‘small talk’ into a conversation with clients.

### Proactive Consultation

The chat bot, functioning as a virtual assistant, is programmed to ‘reach out’ to clients and ask them questions that relate to major life events. Based on the client answers the computer system 1 records the client against one or more demographics. Examples of such questions are whether the client is married, has children or has been to college/university, etc. Based on the client answers, the system 1 builds a profile for each client. For communications with each client the virtual assistant accesses their profile and tailors communications based on the information there. For example the virtual assistant generates an action plan for each client and adopts a different course of communication depending on the demographic that client is in The action plans may involve the system generating communications about the client preparing for a new job, about getting ready to meet someone's family for the first time or about attending their child's wedding. This information is used to give clients more of a feeling that they are dealing with a human, even though they are not. The system 1 is programed so that the action plan for each client is initiated via a conversation trigger while dialoguing with that client. The system 1 is also programmed so that action plans are triggered or created by events, for example detecting that a client has made a purchase off a particular website or the client visiting a specific page or subscribing to a mailing list. The profiles and action plans of users may also be used by the system to identify likely client needs, and to system generate offers to them for related or otherwise relevant goods or services.

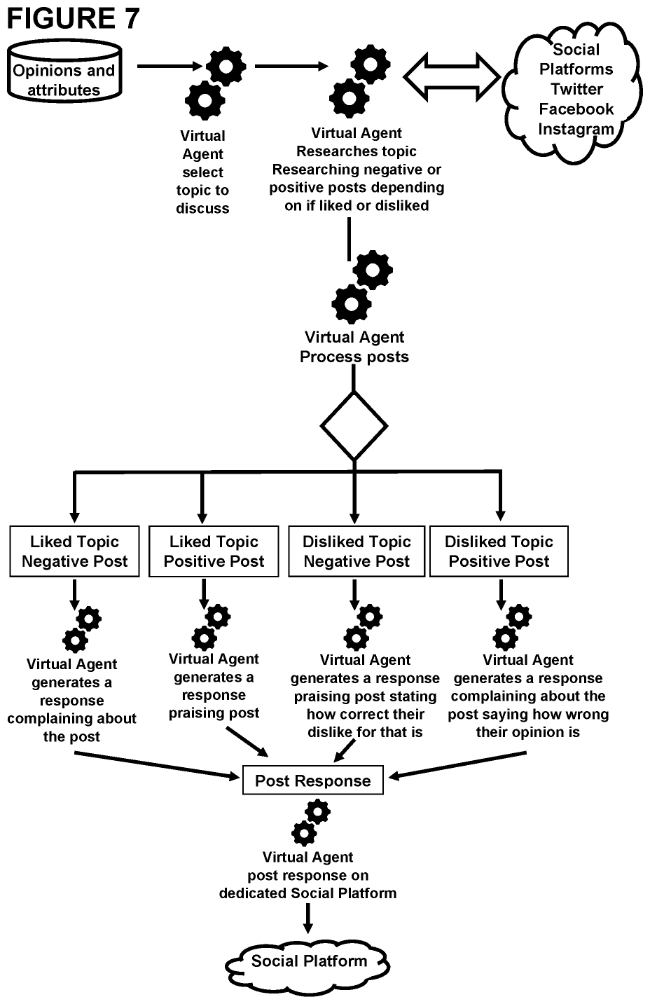

FIG. 7 illustrates a particularly preferred embodiment of the computer system 1, comprising an ‘interests’ data record 3 identifying the interests (also including opinions and attitudes) of clients 2. A software routine or agent 4 accesses the interests data record 3 and identifies matters of interest to each client. The agent then researches the interests, for example based on ‘likes’ or ‘dislikes’ that the client has communicated to the system 1 or on social media platforms such as Twitter, Facebook and Instagram 5. Based on the ‘likes’ or ‘dislikes’ of each client the system 1 generates communications plans for the clients as indicated at items 6a, 6b, 6c and 6d. In the example illustrated, the plan 6a is for communicating with a client after that person has indicated a ‘like’ to a negative post on a social media platform. The plan 6b is for communicating with a client after the person has indicated a ‘like’ to a positive post on a social media platform. The plan 6c is for communicating with a client after the person has indicated a ‘dislike’ to a negative post on a social media platform. The plan 6d is for communicating with a client after the person has indicated a ‘dislike’ to a positive post on a social media platform. The agent 4 then executes each plan by communicating with the client in sympathy to the ‘like’ or ‘dislike’ the client expressed on the social media platform. The communication may be direct, or as a post 8 to the client's social media 5 pages.

In terms of disclosure, this document hereby envisages and discloses each item, step or other feature mentioned herein in combination with one or more of any of the other same or different items, steps or other features disclosed herein, in each case regardless of whether such combination is claimed.

While some preferred forms of the invention have been described by way of example, it should be understood that modifications and improvements can occur without departing from the scope of the following claims.

1. A method of identifying and addressing staff problems in an organisation, comprising the steps of:
using a chat bot to ask a first staff member of the organisation a series of questions pertaining to their wellbeing and to receive the staff member's answers;
computer processing the answers to identify:
that the first staff member has experienced a problem pertaining to their wellbeing in the course of their work for the organisation; and
how the first staff member solved that problem;
using the chat bot to ask a second staff member of the organisation a series of questions pertaining to their wellbeing and to receive the second staff member's answers;
computer processing the second staff member's answers to determine whether that staff member has substantially the same problem that the first staff member had; and
delivering substantially the first staff member's solution to the second staff member.

2. (canceled)

3. A method according to claim 1, wherein the problem, is that the staff member is feeling at least one of:
over-worked;
underutilised;
under-valued;
unappreciated;
pressured;
anxious;
worried;
unknowledgeable;
in need of training;
victimised; and
bullied.

4. A method according to claim 1, wherein the chat bot determines an emotional or mental disposition of the staff members based on the answers they give to the questions.

5. A method according to claim 1, wherein the chat bot receives video image data from communications devices used by the staff members and, based on such data, determines an emotional or mental disposition of the staff members.

6. A method according to claim 5, wherein the staff member's answers, or the video image data, are computer processed to determine whether the staff member in each case is one or more of:
surprised;
confused;
anxious;
agitated;
annoyed;
angry;
sad;
happy;
pleased; and
satisfied.

7. A method according to claim 6, wherein the chat bot communicates with the staff member in a manner sympathetic to the staff member's emotional or mental disposition as determined above.

8. A method according to claim 1, wherein the answers are computer processed to determine a level or performance of the staff members and/or of the organisation they are engaged in.

9. A method according to claim 1, wherein a computer system:
a) records personal circumstances experienced by the staff members and communicated to the system; and
b) chats with the staff members in sympathy with such circumstances.

10. A method according to claim 1, wherein a computer system:
c) identifies ‘likes’ and ‘dislikes’ communicated by the staff members via social media platform accounts of those staff members; and
d) chats with the staff members in sympathy with the likes and dislikes they expressed.
